(19) United States
(12) Patent Application Publication
(10) Pub. No.: US 2023/0181562 A1
(43) Pub. Date: Jun. 15, 2023

(54) USE OF A MULTIFUNCTIONAL LIGAND FOR TREATING DRY EYES, MEIBOMIAN GLAND DYSFUNCTIONS AND LACRIMAL GLAND DYSFUNCTIONS

(71) Applicants: Gaëtan TERRASSE, MONTCEAU-LES-MINES (FR); H4 ORPHAN PHARMA, DIJON (FR)

(72) Inventors: Gaëtan TERRASSE, MONTCEAU-LES-MINES (FR); Catherine BUR, ORGERUS (FR)

(73) Assignees: Gaëtan TERRASSE, MONTCEAU-LES-MINES (FR); H4 ORPHAN PHARMA, DIJON (FR)

(21) Appl. No.: 17/920,916

(22) PCT Filed: Apr. 15, 2021
(86) PCT No.: PCT/EP2021/059813

§ 371 Date: Oct. 24, 2022

(30) Foreign Application Priority Data
Apr. 22, 2020 (FR) FR2004022

(51) Int. Cl. A61K 31/4709; A61K 31/4741; A61K 47/36; A61K 47/32; A61P 27/02

(52) U.S. Cl. CPC A61K 31/4709; A61K 31/4741; A61K 47/36; A61K 47/32; A61P 27/02

(57) ABSTRACT

Use of an isoquinoline multifunctional ligand, amino-7 triethoxy-4,5,6 oxo-1 dihydro-1,3 isobenzofurannyl-3)-1methoxy-8 methyl-2 methylenedioxy 6,7tetrahydro-,2,3,4isoquinoline, or tritoqualine for the treatment of dry eyes, meibomian gland dysfunctions and lacrimal gland dysfunctions.

### FIELD

The present invention relates to the use of a multifunctional ligand and more particularly: the levorotatory and dextrorotatory enantiomers of AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE or tritoqualine for treating dry eye syndrome and dysfunctions of the Meibomian glands, lacrimal glands.

The present invention relates to the use of a multifunctional ligand having activity on the NK1 receptor but also activity on the transmembrane conductance regulator (CFTR) modulator for the treatment of dysfunctions of meibomian glands, lacrimal glands and more generally dry eye syndrome, as well as pharmaceutical compositions, and treatment methods.

### BACKGROUND

Dry eye syndrome is defined by the Dry Eye Workshop in 2007 as a multifactorial disease of the tears and ocular surface leading to symptoms of discomfort, visual disturbance and instability of the tear film with potential damage to the ocular surface. The ocular surface can have more or less severe keratitis which can result in cornea ulcer. It is accompanied by an increase in the osmolarity of the tear film and inflammation of the ocular surface. There are many different aetiologies, and a distinction is conventionally made between dry eye syndromes caused by a decrease in tear flow (such as Gougerot-Sjögren syndrome) and dry eye syndromes caused by tear hyper-evaporation.

Meibomian gland dysfunction (MGD) is the most common cause of dry eye disease (DED). A workshop in 2011 also defined Meibomian gland dysfunction (MGD). Eyelid inflammation, microbial growth, related skin disorders and potentially serious corneal complications result in MGD being a complex multifactorial disorder. Meibomitis can be considered a disease in its own right.

It is likely that MGD is a heterogeneous condition resulting from the combination of the following five distinct pathophysiological mechanisms: eyelid inflammation, conjunctival inflammation, corneal damage, microbiological changes and DED resulting from tear film instability. The pathogenesis of both MGD and DED can be described in terms of a “vicious circle”: the underlying pathophysiological mechanisms of DED and MGD interact, resulting in a double vicious circle. It is this double vicious circle that causes dry eye and the difficulty of treating it.

Dry eye syndrome is a multifactorial and complex ocular surface disease that leads to a loss of tear film homeostasis and causes variable ocular symptoms. The negative effect of DED on visual function, quality of life and economic burden is well recognised (Efficacy of topical ophthalmic drugs in the treatment of dry eye disease: A systematic literature review. Holland E J, et al. Ocul Surf. 2019). In many patients, the disease is chronic and requires long-term treatment.

The prevalence of DED is high, with global estimates ranging from 5 to 50% of the adult population, and the economic burden of the disease is expected to increase as the population ages. Worldwide, ocular lubricants are often used in the initial management of DED, but do not address the underlying causes of the disease.

Potentially more effective ophthalmic pharmacological drugs, targeting different distinct pathophysiological pathways of DED, have been investigated over the past two decades, but these efforts have resulted in the approval of very few new drugs. The main approved treatments are 0.05% cyclosporine A ophthalmic emulsion (Restasis®; Allergan, Irvine, Calif., USA) and 5.0% lifitegrast ophthalmic solution (Xiidra®; Shire, Lexington, USA) in North America, cationic emulsion; Ikervis®; Santen Pharmaceutical, Osaka, Japan) in Europe and 3% diquafosol based ophthalmic solution (Diquas®; Santen Pharmaceutical, Osaka, Japan) and a unit dose of 2% rebamipide ophthalmic suspension (Mucosta®; Otsuka Pharmaceutical, Tokyo, Japan) in Asia In the USA (August 2018), a 0.09% Cyclosporine A nano-micellar formulation (Cequa®; Sun Pharmaceuticals, Mumbai, India) has been approved to increase tear production in patients with DED. Overall, these drugs reduce ocular surface inflammation or stabilise the tear film, although it is unknown which drugs are most suitable for patients with Aqueous Deficient Dry Eye (ADDE) or evaporative Dry Eye (EDE). Gougerot-Sjögren's syndrome (GSS) is associated with xerophthalmia due to progressive destruction of the lacrimal glands, which can be responsible for severe keratitis. GSS is a chronic autoimmune condition characterised by progressive, degenerative, inflammatory involvement of the exocrine glands, which may be associated with systemic disease that variably affects joints, skin, lungs, kidneys or peripheral nerves. The pathophysiology of the disease is characterised by infiltration of the salivary and lacrimal glands by CD4+ T cells and B cells. The local activation and proliferation of these lymphocytes triggers the release of pro-inflammatory cytokines that maintain a state of chronic inflammation, as well as the secretion of autoantibodies, ultimately leading to death by apoptosis of the epithelial cells.

The usual and initial management of dry eye syndrome, whatever the aetiology, is based on:

the correction of promoting factors, as far as possible (drugs, environmental factors, eye drops containing preservatives, in particular quaternary ammoniums);

and replacement therapy with tear substitutes [artificial tears in eye drops, gels, as well as medical devices of viscoelastic solutions used after failure of the other two].

Once initiated, dry eye may progress despite replacement therapy and become self-maintaining according to the concept of the vicious circle of inflammation with progressive damage to all ocular surface tissues, including the cornea. In severe forms, dry eye can lead to major damage to the cornea [or keratitis], with a range of symptoms from a sensation of a foreign body on the ocular surface and burning to permanent pain with reduced visual acuity. The severity of dry eye is related to the extent of keratitis, inflammatory component and painful ocular symptoms.

Several pharmacological targets need to be treated to treat causes of the vicious circles responsible for dry eye. This is what multifunctional ligands do. A multifunctional ligand, as the name suggests, is active on several pharmacological targets. To be effective, multifunctional ligands have a lower affinity on the receptors, allowing them to diffuse in a balanced way on several pharmacological targets. Their affinity is more likely to be between 100 nanomolar and 1 micromolar. The inventors have demonstrated that tritoqualine behaves as a multifunctional ligand, with activity on two drug targets, CFTR and NK1.

CFTR is a multifunctional protein. The CFTR protein is a member of the ABC transporter superfamily, which is found in all domains of life (bacteria, archaea and eukaryotes). It is distinguished from all other members of this superfamily by its status as an ion channel as well as by the presence of its unique regulatory domain (R). It forms a channel permeable to chloride and thiocyanate ions in epithelial cells. Other functions, independent of channel regulation, have been described: ATP transport, modulation of exocytosis/endocytosis phenomena, regulation of pH of intracellular organelles.

In dry eye syndrome it is necessary to restore the production of tears from the lacrimal glands, but also the secretion of electrolytes from the corneal epithelium.

It was demonstrated in a paper published in 2001 that CFTR had a role in electrolyte secretion in an immortalised rabbit corneal epithelial cell line (Invest Ophthalmol Vis Sci. 2001 Sep Activation of a CFTR-mediated chloride current in a rabbit corneal epithelial cell line. Al-Nakkash L). But also on lacrimal gland secretion (Invest Ophthalmol Vis Sci. 2018 Jan Novel Insight Into the Role of CFTR in Lacrimal Gland Duct Function in Mice. Berczeli O.). In this paper the authors show that a CFTR modulator is able to increase tear secretion in mice with normal CFTR.

CFTR is a cAMP/ATP-mediated anion channel that is expressed in a variety of cell types, including secretory epithelial cells, where it regulates anion flow across the membrane, as well as the activity of other ion channels and proteins. In epithelial cells, normal CFTR function is essential for maintaining electrolyte transport throughout the body, including respiratory and digestive and even eye tissues. CFTR is comprised of approximately 1480 amino acids making up a protein comprised of a repeat of transmembrane domains, each containing six transmembrane helices and a nucleotide binding domain. The two transmembrane domains are connected by a large polar regulatory domain [R] with several phosphorylation sites that regulate channel activity and cell trafficking.

Chloride transport occurs through the coordinated activity of ENaC and CFTR present on the apical membrane and the Na+ and K+ ATPase and Cl-channels expressed on the basolateral surface of the cell. Secondary active chloride transport from the lumen side leads to the accumulation of intracellular chloride, which can then passively leave the cell through Cl-channels, resulting in transport from the basal pole to the apical pole.

Thus water, which is probably never actively transported, is transported across the epithelia with transepithelial osmotic gradients generated by the flow of sodium and chlorides. There is a need to find new therapies to modulate and activate normal CFTR function in order to treat the cause of dry eye syndrome which appears to be impacted by insufficient CFTR function.

On the other hand, recent evidence suggests that some dry eye symptoms may be better represented by a chronic neuropathic disorder.

Neurogenic mechanisms may play an important role in chronic ocular surface inflammation. Manifestations may be associated with repeated ocular sensory nerve damage leading to an acute to chronic transition associated with neuropathological changes. These neuropathic changes are also the origin of dry eye pain.

Substance P is thought to be involved in this neurogenic inflammation. Stern ME and colleagues (The Pathology of Dry Eye: The Interaction Between the Ocular Surface and Lacrimal Glands, Cornea 1998 November).

While neurogenic inflammation is not fully understood in dry eye syndrome, it is certainly involved in an important way (J Investig Allergol Clin Immunol. 2018 Sep Neuropathic Pain and Itch Mechanisms Underlying Allergic Conjunctivitis. Kuruvilla M et al).

Thus, it would be interesting both to modulate CFTR and inhibit substance P to break the vicious cycle of dry eye syndrome. Substance P is secreted after activation of the NK1 receptor. The multifunctional ligand that is represented by tritoqualine acts as an NK1 antagonist and thus a substance P secretion inhibitor. WO2007/117704A2 discloses tritoqualine in the treatment of an immune system disease such as conjunctivitis.

Holland et al. (OCULAR SURFACE, vol. 17, no. 3, 1 Jul. 2019, pages 412-423) describes different topical compounds for the treatment of dry eye syndrome.

Flores et al. (The FASEB Journal, vol. 30, no. 5, 1 May 2016, pages 1789-1797) discloses small molecule CFTR activators, which increase tear secretion and prevent dry eye syndrome, in particular including Sjögren's syndrome, but does not disclose tritoqualine.

EP2659890A1 discloses the use of tritoqualine in the treatment of fibrotic diseases and in particular cystic fibrosis.

### SUMMARY

The present invention relates to the use of a multifunctional ligand, in particular comprising isoquinoline-based compounds including levorotatory and dextrorotatory enantiomers of AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE or tritoqualine and pharmaceutically acceptable salts thereof.

The inventors have discovered surprising properties of AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof as a multifunctional ligand acting on the normal CFTR modulation and the NK1 receptor.

The invention also relates to pharmaceutical compositions comprising at least one of the compounds described herein and/or at least one pharmaceutically acceptable salt thereof, which compositions may further comprise at least one other active pharmaceutical ingredient and/or at least one excipient. The invention also relates to methods for treating dry eye syndrome, as well as Gougerot-Sjögren's disease, consisting in administering at least one of the compounds described herein and/or at least one pharmaceutically acceptable salt thereof, optionally as part of a pharmaceutical composition comprising at least one additional component, to a subject in need thereof.

Tritoqualine is known to have anti-allergic activity through its inhibitory action on histidine decarboxylase. However, this activity is very weak and does not explain numerous properties it has on various clinical symptoms, rhinitis, urticaria, eczema, mastocytosis.

The inventors have demonstrated that tritoqualine has a very significant action on CFTR and the NK1 receptor, two important targets for treating dry eye. Many patents have been filed based on tritoqualine, but none of them mention its activity in the treatment of dry eye syndrome.

Tritoqualine is a benzylisoquinoline with a molecular weight of 500. This compound can be modified or substituted with compounds comprising either carbon-14 or deuterated compounds.

Isotope-labelled compounds and salts can be used in a variety of ways. They may be suitable for drugs and/or different types of tests, such as tissue distribution tests on a substrate. For example, tritium and/or carbon-14 labelled compounds are particularly useful for various types of tests, such as substrate-based tissue distribution tests, due to their relatively simple preparation and excellent detectability. For example, deuterium labelled products are therapeutically useful and have potential therapeutic advantages over non-deuterium labelled compounds. In general, deuterium-labelled compounds and salts can have higher metabolic stability than non-deuterium-labelled compounds due to the isotope kinetic effect. Higher metabolic stability translates directly into an increased in vivo half-life or lower doses, which could be desired. Isotopically labelled compounds and salts can generally be prepared by following the procedures described in known synthesis schemes such as patent EP3352757. It is thus easy to replace non-deuterated methyls with deuterated methyls.

Tritoqualine is a white crystalline powder. Insoluble in water; sparingly soluble in benzene and acetone.

### BRIEF DESCRIPTION OF THE FIGURES

FIG. 1 represents the chemical structure of tritoqualine.

FIG. 2 Activity of tritoqualine at the apical pole expressed as % of maximum effect.

FIG. 3 Activity of tritoqualine at the basal pole expressed as % of maximum effect.

FIG. 4 Effect of Tritoqualine expressed as μA/Cm² at the apical pole on lacrimal gland epithelial cells (A=Forskolin; B=Tritoqualine; C=Inh 172).

FIG. 5 Effect of Tritoqualine expressed in μA/Cm² at the basal pole on lacrimal gland epithelial cells (A=Forskolin; B=Tritoqualine; C=Inh 172).

FIG. 6 The lacrimal functional unit according to DARTT 2002.

FIG. 7 Affinity curve of tritoqualine on the NK 1 receptor.

FIG. 8 Inhibition percentage of the NK 1 receptor by tritoqualine.

FIG. 9 Flow cytometry analysis highlighting basophil degranulation by substance P (CD63+ and CCR3+).

FIG. 10 Inhibition of basophil degranulation by (10 μM) tritoqualine via inhibition of (10 μM) substance P action.

### DETAILED DESCRIPTION

The inventors have highlighted amazing and surprising properties of tritoqualine in a human cell model of CFTR modulation without mutation.

Thus, the present invention relates to AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof for use in the treatment of diseases associated with decreased secretions of epithelial cells of the lacrimal glands and Meibomian glands.

Thus, the present invention also relates to AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof for use in the treatment of dry eye syndrome.

Thus, the present invention also relates to AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof for use in the treatment of dry eye syndrome related to Gougerot-Sjögren's disease and secondary Gougerot-Sjögren's syndromes accompanying autoimmune diseases such as rheumatoid arthritis or systemic lupus erythematosus.

According to one preferred embodiment, the invention relates to AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof for use in the treatment of pain in dry eye syndrome.

According to one preferred embodiment, the invention relates to AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof for use in dry eye syndrome characterised in that it is administered in the form of an eye drop or eye ointment. According to one preferred embodiment, the invention relates to AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof for use in dry eye syndrome characterised in that it is administered in the form of an eye drop at a dose of 0.1 milligram to 5 milligrams.

According to one preferred embodiment, the invention relates to AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof for use in dry eye syndrome characterised in that it is administered in the form of an eye drop in association with humectants and lubricants based on hyaluronic acid or carbomers. According to one preferred embodiment, the invention relates to AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof for use in the treatment of dry eye syndrome characterised in that it is substituted at at least one of its methyls with a deuterated methyl.

The present invention also relates to AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof for use in improving ion transport both apically and at the basal pole of corneal, lacrimal glandular and meibomian glandular epithelial cells.

According to the preferred embodiment of the invention AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof is remarkable in that it is substituted at its methyls with deuterated methyls to improve pharmacokinetics.

According to one preferred embodiment of the invention, AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof is for use in the treatment of dry eye syndrome at a dose of 0.1 milligram to 5 milligrams/day.

According to one preferred embodiment of the invention, AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof also for use in the treatment of dry eye syndrome is remarkable in that it is packaged in the form of an eye drop or eye ointment.

### EXAMPLES

To investigate this action on CFTR modulation, the inventors used the Ussing chamber method, invented by the Dane Hans H. Ussing in the late 1950s. This technique makes it possible to study ionic exchanges across an epithelium, as it allows the tissue of interest to be kept alive for a few hours under controlled conditions of temperature and medium. The positioning of the tissue between two half-chambers makes it possible to define an apical compartment (corresponding to the lumen of the organ) and a basolateral compartment (corresponding to the blood compartment) and to investigate exchanges between these two compartments via the tissue.

This technique is used as a routine and is particularly well adapted for a pharmacological approach to ionic transport and to search for molecules of therapeutic interest in the context of ionic secretions of epithelial cells. It consists in measuring the transepithelial current (known as the short-circuit current and noted Ise). Ise is expressed in amperes to unit area of the epithelium (Ise in μA/cm²). The cells are grown on a porous filter for 10 to 15 days at the liquid-liquid interface and then air-liquid interface in order to mimic conditions close to in vivo. The transepithelial resistance is measured regularly when culturing. The higher this resistance is (several hundred ohms), the more the epithelial tissue is junctive, polarised and therefore tight. At the air (apical side)-liquid (basolateral side) interface the epithelial cells polarise and form a tight mat which can be investigated with the Ussing chamber technique. A system with 6 Physiology Instrument® cuvettes allowing 6 experiments in parallel has been used.

The following material and molecules have been used in the study:

Amiloride: 100 μM final concentration; 100 mM stock solution, solvent water (supplier Sigma®)).

Forskolin: 0.05 μM final concentration; 1 mM stock solution, DMSO solvent (supplier Sigma®)).

Genistein: 30 μM final concentration; 30 mM stock solution, DMSO solvent (supplier Sigma®)).

CFTR inh172: 10 μM final concentration; 10 mM stock solution, DMSO solvent (supplier Fisher®).

UTP: 100 μM final concentration; 100 mM stock solution, DMSO solvent (supplier SigmaC).

Various media and reagents including supports adapted to the Ussing chamber: Snapwell (Fisher®), culture medium (Gibco®), SVF (Gibco®), Puromycin (Gibco®), T75 culture flasks (FisherED) have been used.

Tritoqualine has a surprising action on CFTR, which is not known. No publication or patent mentions any activity of tritoqualine on CFTR of corneal epithelial cells or glandular epithelial cells.

The inventors have used USSING chambers to analyse the activity of tritoqualine on CFTR of glandular epithelial cells.

Typical protocols used for human epithelial cells expressing non-mutated CFTR:

Measurement of short circuit current in the presence of amiloride (ENaC channel inhibitor, 100 μM) followed by addition of 10 μM of the tritoqualine molecule and then addition of CFTR inh172 (10 μM, CFTR inhibitor) followed by addition of UTP (100 μM, validates the experiment by activating the calcium-sensitive Cl transport).

Measurement of short circuit current in the presence of amiloride (100 μM) and forskolin (0.05 μM activator of intracellular cAMP) then addition of 10 μM of the tritoqualine molecule then addition of CFTRinh172 (10 μM) then addition of UTP (100 μM).

Measurement of the short circuit current in the presence of CFTR Inh172 (10 μM) then addition of tritoqualine and forskolin (0.05 μM) then addition of UTP.

The inventors have prepared a 100 μM stock solution in DMSO. The compounds have been aliquoted per 100 μL and stored at −20° C.

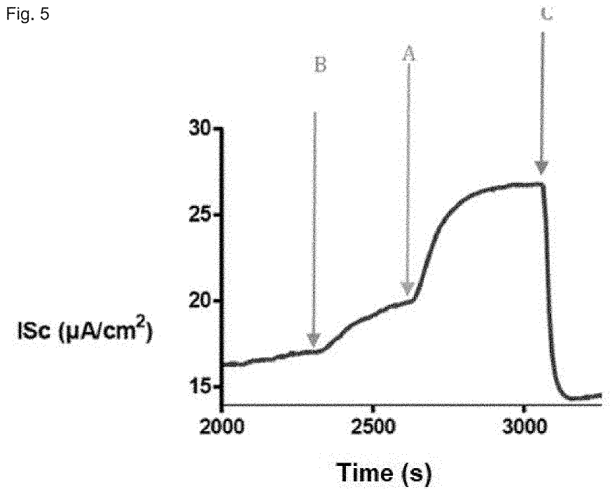

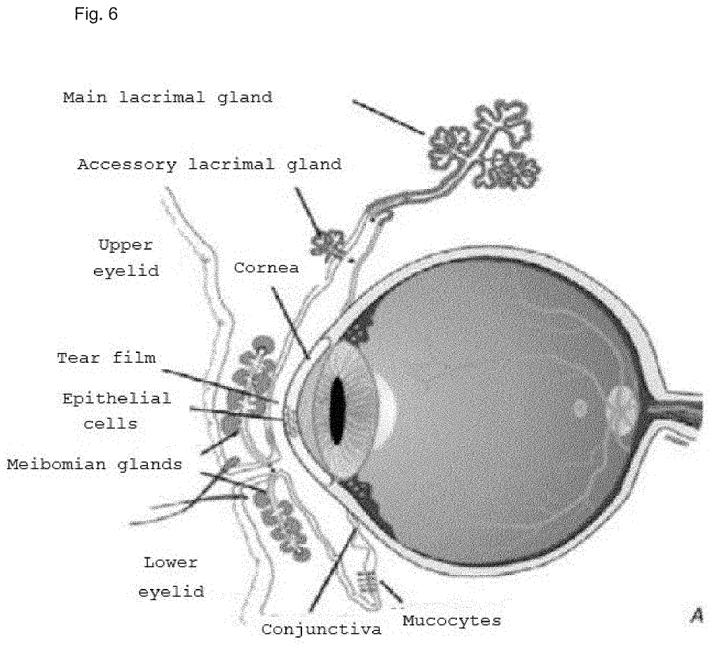

Tritoqualine has been added to USSING cells at a dose of 10 μM in non-mutated cells FIG. 5.

The effect of tritoqualine on non-mutated epithelial cells on the apical side is significant, as the differential increases from 16 to more than 20 (expressed in μA/cm²).

The addition of forskolin changes the potential from 20 to 27 (expressed in μA/cm²).

The addition of Inh172 completely blocks the cell potential.

Tritoqualine activates the cell potential in lacrimal gland epithelial cells expressing non-mutated CFTR. This effect is additive with that of forskolin.

The effect of tritoqualine on non-mutated lacrimal gland epithelial cells on the apical side is significant, as the differential increases from 6.5 to more than 7.5 (expressed in μA/cm²). The addition of forskolin changes the potential from 3.5 to 6.5 (expressed in μA/cm²).

The addition of Inh172 completely blocks the cell potential.

In conclusion, tritoqualine activates ion transport in the epithelial cells of the lacrimal gland. This effect is blocked by inh172, which specifically inhibits CFTR ion transport.

Tritoqualine appears to be a molecule capable of stimulating ion transport via the non-mutated CFTR.

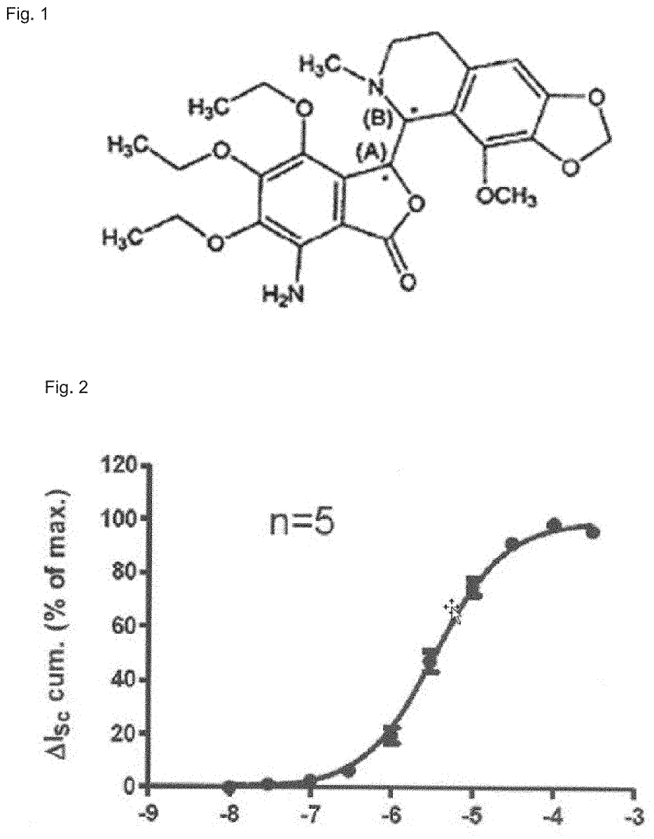

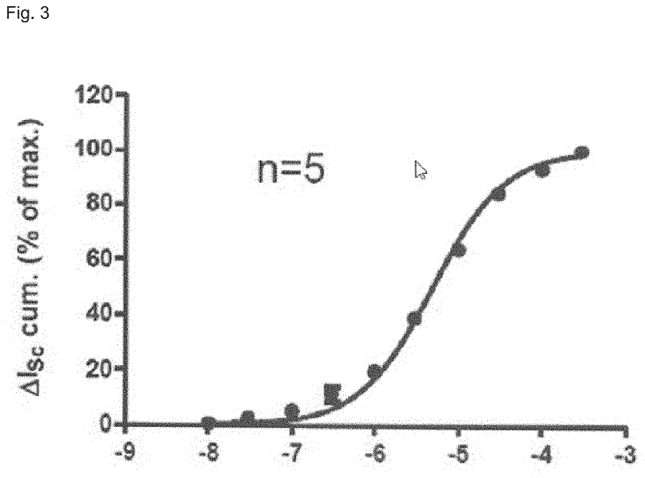

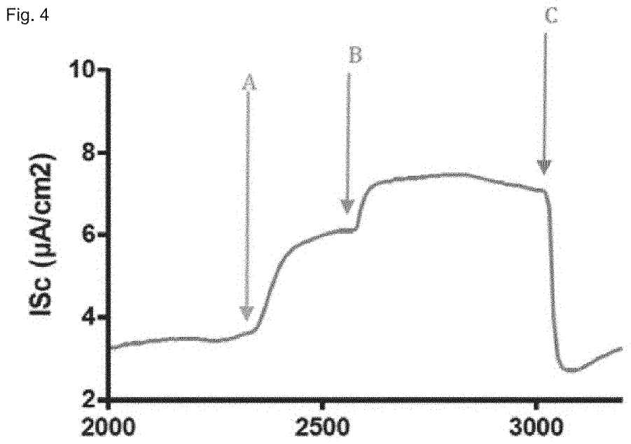

The effective dose of tritoqualine has then been determined on both the apical and basal poles. The result is an EC50 activity of 3.42+/−0.19 μM for the apical pole and an EC50 of 4.87+/−0.27 μM for the basal pole FIG. 2, FIG. 3.

Thus, tritoqualine can improve the lacrimal secretion function of patients with proven dry eye disease including patients with Gougerot-Sjögren's disease, but also so-called “secondary” Gougerot-Sjögren's because they are associated with specific autoimmune diseases such as rheumatoid arthritis or systemic lupus erythematosus.

The inventors have also used an NK 1 agonist (substance P—Supplier: Sigma-Aldrich) to demonstrate the activity of tritoqualine on the NK 1 receptor.

Tritoqualine has been tested on the NK 1 receptor according to the method described by Heuillet et al., Characterization of a human NK1 tachykinin receptor in the astrocytoma cell line U 373 MG. Heuillet E et al. Neurochem. 1993 March.

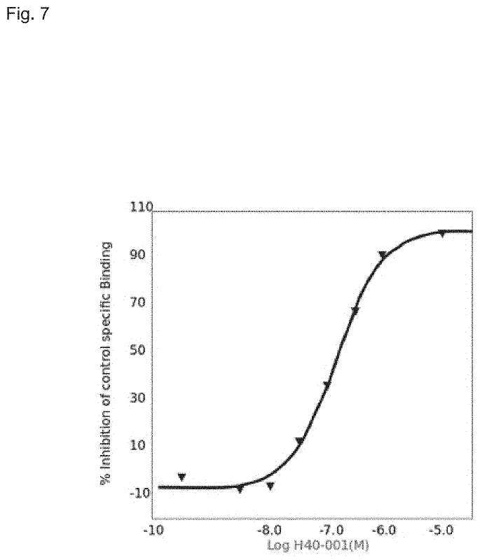

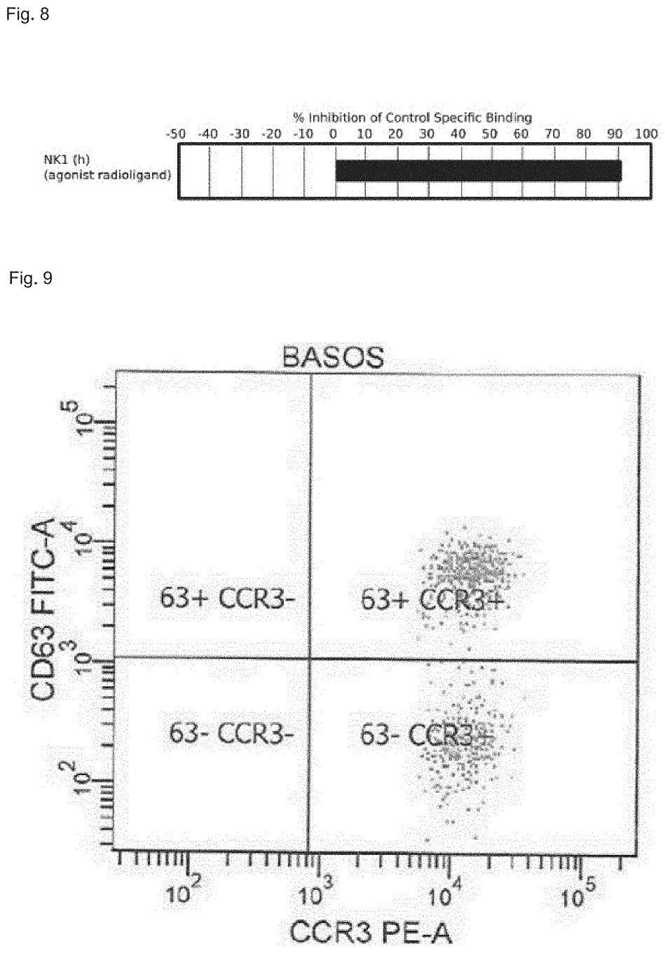

This test shows an affinity of tritoqualine, which is equal to 97% and a Ki equal to 1.4 10-7. FIG. 7 shows precisely the affinity curve and Ki of tritoqualine on the NK 1 receptor. FIG. 8 shows the percent of affinity with the NK 1 receptor. Tritoqualine appears as an NK 1 receptor antagonist in this experiment. It has been verified that the NK 1 receptor was involved in the basophil activation pathway by using an NK 1 agonist.

The inventors have used the Kit-Flow CAST (www.buhlmannlabs.ch/products-solutions/cellular-allergy/flow-cast/) to test the action of tritoqualine on the inhibition of basophil degranulation. The marker for degranulation is CD63. Upon basophil activation, CD63 markers bound to intracytoplasmic granules will fuse with the plasma membrane. They are then expressed on the cell surface: activated basophils thereby become CD63+. Further to CD63, another marker specific to basophils makes it possible to better target them, it is CCR3 (chemokine receptor 3). The latter is always expressed by this cell type. Thus, the degranulation of basophils is identified in the flow cytometry window as CD63+ and CCR3+.

In a first step 2 patients have been sampled. substance P has been tested at a dose of 10 μmoles. In this experiment, it is surprisingly found that CD63 is present at 70% on the basophil. FIG. 9 shows the intensity of degranulation when substance P is incubated with basophils implying that substance P caused degranulation of the basophil.

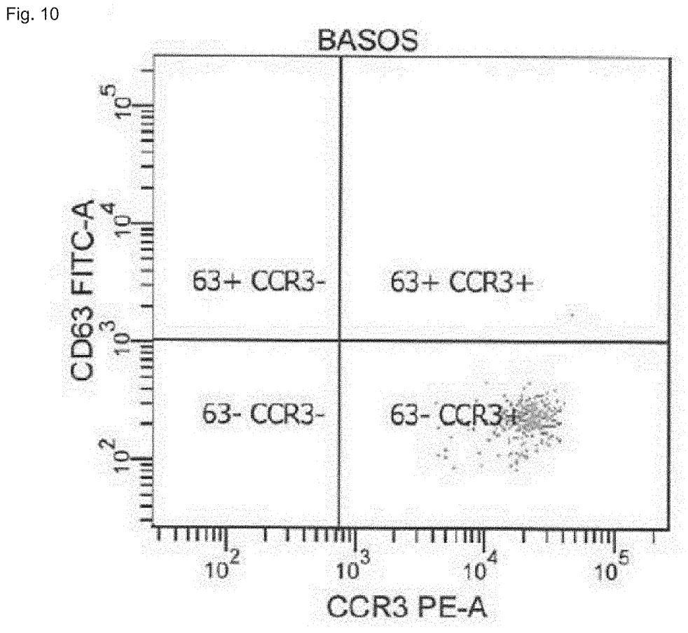

In a second step to determine whether tritoqualine also blocked substance P-induced degranulation, the following experiment has been methodically performed: tritoqualine has been incubated at a dose of 10 pmoles without the FcεRI antibody and then substance P has been put into the tubes. After 30 minutes, degranulation in each of the tubes is around 7% as with the negative control. FIG. 10 shows that tritoqualine blocks the activation of basophil degranulation caused by substance P. It can be said that tritoqualine has a surprising action on the inhibition of degranulation via its antagonistic action on the NK 1 receptor.

Thus, in the dry eye, tritoqualine would have 2 actions via the inhibition of substance P: an action on the basophil and an action on the neurological vicious circle of the dry eye. Thus tritoqualine, through its action on NK1, would have 2 complementary actions; one on the inflammation related to the basophil degranulation inhibition and another on the pain related to its action on the neurological vicious circle. Thus, the local action of tritoqualine on dry eye appears remarkable and surprising due to its 2 pharmacological actions on CFTR and substance P.

1-7. (canceled)

8. A method of treating dry eye syndrome comprising administering to a subject in need thereof an effective amount of AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof.

9. The method according to claim 8, wherein the effective amount of AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof treats pain in dry eye syndrome.

10. The method according to claim 8, wherein the effective amount of AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof treats dry eye syndrome related to Gougerot-Sjögren's disease.

11. The method according to claim 8, wherein the effective amount of AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof treats dry eye syndrome related to autoimmune diseases such as rheumatoid arthritis or systemic lupus erythematosus.

12. The method according to claim 8, wherein the AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof is administered in combination with carbomer-based humectants or hyaluronic acid-based lubricants.

13. The method according to claim 8, wherein the AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof is substituted at at least one of its methyls with a deuterated methyl.

14. The method according to claim 8, wherein the AMINO-7 TRIETHOXY-4,5,6 OXO-1 DIHYDRO-1,3 ISOBENZOFURANNYL-3)-1METHOXY-8METHYL-2METHYLENEDIOXY-6,7TETRAHYDRO-,2,3,4ISOQUINOLINE, or tritoqualine and pharmaceutically acceptable salts thereof is packaged in the form of an ophthalmic eye drop or ointment.
